(19) United States
(12) Patent Application Publication
(10) Pub. No.: US 2004/0137526 A1
(43) Pub. Date: Jul. 15, 2004

(54) Multidimensional protein separation system

(75) Inventors: Samir M. Hanash, Ann Arbor, MI (US); Hong Wang, Ann Arbor, MI (US)

Correspondence Address: Tanya A. Arenson, MEDLEN & CARROLL, LLP, Suite 350, 101 Howard Street, San Francisco, CA, 94105, US

(73) Assignee: The Regents of the University of Michigan, Ann Arboro, MI

(21) Appl. No.: 10/686,417

(22) Filed: Oct. 15, 2003

Related U.S. Application Data
(60) Provisional application No. 60/418,885, filed on Oct. 15, 2002.

(51) Int. Cl. G01N 33/53; G01N 33/557

(52) U.S. Cl. 435/7.1; 436/517

(57) ABSTRACT

The present invention relates to systems, apparatuses and methods for multidimensional protein separation. In particular, the present invention relates to 3-dimension protein separation and characterization systems and methods.

This application claims priority to provisional patent application serial No. 60/418,885, filed Oct. 15, 2002, which is herein incorporated by reference in its entirety.

### FIELD OF THE INVENTION

The present invention relates to systems, apparatuses and methods for multidimensional protein separation. In particular, the present invention relates to 3-dimension protein separation and characterization system and methods.

### BACKGROUND OF THE INVENTION

As the nucleic acid sequences of a number of genomes, including the human genome, become available, there is an increasing need to interpret this wealth of information. While the availability of nucleic acid sequence allows for the prediction and identification of genes, it does not explain the expression patterns of the proteins produced from these genes. The genome does not describe the dynamic processes on the protein level. For example, the identity of genes and the level of gene expression do not represent the amount of active protein in a cell nor does the gene sequence describe post-translational modifications that are essential for the function and activity of proteins. Thus, in parallel with the genome projects there has begun an attempt to understand the proteome (i.e., the quantitative protein expression pattern of a genome under defined conditions) of various cells, tissues, and species. Proteome research seeks to identify targets for drug discovery and development and provide information for diagnostics (e.g., tumor markers).

An important area of research is the study of the protein content of cells (e.g., the identity of and amount of expressed proteins in a cell). This field requires methods that can separate large numbers of proteins and can do so quantitatively so that changes in expression or structure of proteins can be detected. The method generally used to achieve such cellular protein separations is 2-D PAGE. This method is capable of resolving hundreds of proteins based upon pI in one dimension and protein size in the second dimension. The proteins separated by this method are visualized using a staining method that can generally be quantified. The result is a 2-dimensional image where the protein map is based on pI and approximate molecular weight. By the use of computer based image analysis techniques, one can search for proteins that are differentially expressed in various cell lines. These methods are used to monitor changes in protein expression that are linked to conditions such as cell transformation and cancer progression, cell aging, the response of cells to environmental insult, and the response of cells to pharmaceutical agents. Once changes in protein expression have been identified, then one can further analyze target proteins to determine their identity and whether they have been altered from their expected structure by sequence changes or post-translational modifications.

Although 2-D PAGE is still widely used for protein analysis, the method has several limitations, including the fact that it is labor intensive, time consuming, difficult to automate and often not readily reproducible. In addition, quantitation, especially in differential expression experiments, is often difficult and limited in dynamic range. Also, while the 2-D gel does produce an image of the proteins in the cell, the mass determination is often only accurate to 5-10%, and the method is difficult to interface to mass spectrometric techniques for further analysis.

Another limitation of 2-D PAGE is the amount of protein loaded per gel, which is generally below 250 μg. The amount of protein in any given spot may therefore be too low for further analysis. For Coomassie brilliant blue (CBB) stained gels the limit of detection is 100 ng per spot while for silver stained gels the limit of detection is 1-10 ng. Furthermore, proteins that have been isolated in 2-D gels are embedded inside the gel structure and are not free in solution, thus making it difficult to extract the protein for further analysis. Because of these limitations, the art is in need of protein mapping methods that are efficient, automated, and have broader resolution capabilities than presently available technologies.

### SUMMARY OF THE INVENTION

The present invention relates to systems, apparatuses and methods for multidimensional protein separation. In particular, the present invention relates to 3-dimension protein separation and characterization system and methods.

Accordingly, in some embodiments the present invention provides a method, comprising providing a sample comprising a plurality of polypeptides; a first separation device configured for separation of the polypeptides in the sample based on charge; a second separation device configure for separation of the polypeptides is the sample based on hydrophobicity; and a third separation device configured for separation of the polypeptides in the sample based on size; and separating the sample with the first separation device to generate a charge separated protein sample, wherein the charge separated sample comprises a plurality of fractions; separating the charge separated sample with the second separation device to generate a charge and hydrophobicity separated sample, wherein the charge and hydrophobicity separated sample comprises a plurality of fractions; and separating the charge and hydrophobicity separated sample with the third separation device to generate a charge, hydrophobicity, and size separated sample, wherein the charge, hydrophobicity and size separated sample comprises a plurality of fractions. In some embodiments, the first separation device is configured for performing a separation technique including, but not limited to, isoelectric focusing gel electrophoresis, free-flow electrophoresis, rotofor electrophoresis and ion exchange chromatography. In some embodiments, the second separation device is configured for performing a separation technique including, but not limited to, reversed-phase chromatography and hydrophobic interaction chromatography. In some embodiments, the third separation device is configured for performing a separation technique including, but not limited to, SDS-gel electrophoresis, size exclusion chromatography, and capillary electrophoresis. In some embodiments, the method further comprises the step of detecting polypeptides in the fractions of the charge, hydrophobicity, and size separated sample. In some embodiments, the detecting comprises a detection method including, but not limited to, UV/VS spectrophotometry, fluorescence spectrophotometry, and mass spectrometry. In some embodiments, the mass spectroscopy is selected from the group including, but not limited to, MALDI-TOF-MS, ESI oa TOF, ion trap mass spectrometry, ion trap/time-of-flight mass spectrometry; quadrupole mass spectrometry, triple quadrupole mass spectrometry, Fourier Transform (ICR) mass spectrometry, and magnetic sector mass spectrometry. In some embodiments, the method further comprises the step of attaching the plurality of fractions of the charge, hydrophobicity, and size separated sample to a solid support. In some embodiments, the plurality of fractions are arrayed on the solid support. In some embodiments, the method further comprises the step of performing a functional assay on the arrayed plurality of fractions. In some embodiments, the functional assay comprises an antibody binding assay. In certain embodiments, the plurality of polypeptide comprise a proteome.

In some embodiments, the method further comprises further providing a second sample comprising a plurality of polypeptides. In some embodiments, the sample comprises a proteome of a non-cancerous cell and the second sample comprises a proteome of a cancerous cell. In some embodiments, the method further comprises the step of comparing the charge, hydrophobicity, and size separated sample to a charge, hydrophobicity, and size separated second sample.

The present invention further provides a protein separation apparatus, comprising a first separation device, wherein the first separation device is a protein charge separation device; a second separation device, wherein the second device is a protein hybrophobicity separation device; and a third separation device, wherein the third separation device is a protein size separation device. In some embodiments, the first separation device is selected from the group including, but not limited to, an isoelectric focusing gel electrophoresis device, a free-flow electrophoresis device, a rotofor electrophoresis device, and an ion exchange chromatography device. In some embodiments, the second separation device is selected from the group including, but not limited to, a reversed-phase chromatography device and a hydrophobic interaction chromatography device. In some embodiments, the third separation device is selected from the group including, but not limited to, an SDS-gel electrophoresis device, a size exclusion chromatography device, and a capillary electrophoresis device. In some embodiments, the apparatus further comprises a detection device. In some embodiments, the detection device is selected from the group including, but not limited to, a UV/VS spectrophotometer, a fluorescence spectrophotometer, and a mass spectrometer. In some embodiments, the mass spectrometer is selected from the group including, but not limited to, a MALDI-TOF-MS, a ESI oa TOF, an ion trap mass spectrometer, an ion trap/time-of-flight mass spectrometer; a quadrupole mass spectrometer, a triple quadrupole mass spectrometer, a Fourier Transform (ICR) mass spectrometer, and a magnetic sector mass spectrometer.

The present invention also provides a system comprising a protein separation apparatus, the apparatus comprising a first separation device, wherein the first separation device is a protein charge separation device; a second separation device, wherein the second device is a protein hybrophobicity separation device; and a third separation device, wherein the third separation device is a protein size separation device. In some embodiments, the first separation device is selected from the group including, but not limited to, a isoelectric focusing gel electrophoresis device, a free-flow electrophoresis device, a rotofor electrophoresis device, and an ion exchange chromatography device. In some embodiments, the second separation device is selected from the group including, but not limited to, a reversed-phase chromatography device and a hydrophobic interaction chromatography device. In some embodiments, the third separation device is selected from the group including, but not limited to, an SDS-gel electrophoresis device, a size exclusion chromatography device, and a capillary electrophoresis device. In certain embodiments, the apparatus further comprises a detection device. In some embodiments, the detection device is selected from the group including, but not limited to, a UVNS spectrophotometer, a fluorescence spectrophotometer, and a mass spectrometer. In some embodiments, the mass spectrometer is selected from the group including, but not limited to, a MALDI-TOF-MS, a ESI oa TOF, an ion trap mass spectrometer, an ion trap/time-of-flight mass spectrometer; a quadrupole mass spectrometer, a triple quadrupole mass spectrometer, a Fourier Transform (ICR) mass spectrometer, and a magnetic sector mass spectrometer. In certain embodiments, the system further comprises a protein characterization apparatus in communication with the protein characterization apparatus. In some embodiments, the protein characterization apparatus is a protein array analysis apparatus. In some embodiments, the protein array analysis apparatus is configured for performing a functional assay (e.g., an antibody binding assay) on a separated protein sample.

### DESCRIPTION OF THE FIGURES

FIG. 1 shows examples of first dimension separation of three different cell lysates using anion exchange chromatography.

FIG. 2 shows examples of second dimension reversed phase separations of individual fractions from the first dimension separation shown in FIG. 1.

FIG. 3 shows examples of second dimension reversed phase separations following first dimension liquid iso-focusing separation.

FIG. 4 shows an example of a third dimension separation of plasma protein fractions obtained from a reversed phase second dimension and a liquid isoelectric-focusing first dimension.

FIG. 5 shows an example of a third dimension separation of plasma protein fractions obtained from a reversed phase second dimension and a liquid isoelectric-focusing first dimension.

FIG. 6 shows an example of a third dimension separation of plasma protein fractions obtained from a reversed phase second dimension and a liquid isoelectric-focusing first dimension.

FIG. 7 shows an example of a third dimension separation of plasma protein fractions obtained from a reversed phase second dimension and a liquid isoelectric-focusing first dimension.

FIG. 8 shows an example of a third dimension separation of plasma protein fractions obtained from a reversed phase second dimension and a liquid isoelectric-focusing first dimension.

FIG. 9 shows an example of a third dimension separation of plasma protein fractions obtained from a reversed phase second dimension and a liquid isoelectric-focusing first dimension.

FIG. 10 shows an example of plasma proteins separated using the methods of some embodiments of the present invention and identified using mass spectroscopy.

FIG. 11 shows an additional example of plasma proteins separated using the methods of some embodiments of the present invention and identified using mass spectroscopy.

### GENERAL DESCRIPTION OF THE INVENTION

For many years, the large-scale profiling of gene expression at the protein level has largely relied on the availability of procedures for one-dimensional or two-dimensional (2-D) separation of protein mixtures (Hanash, Electrophoresis 21:1202 [2000]). In the past decade, mass spectrometry has facilitated the identification, through sequence database searching, of proteins separated by 2-D gels or other means, at an unprecedented level of sensitivity and speed (Patterson, Physiol. Genomics 2:59 [2000]). The 2-D gel procedure as commonly practiced consists of protein charge based separation in the first dimension that achieves partial resolution of proteins based on their pI and further separation in the second dimension based on the molecular weight of proteins. A major limitation of approaches to profile tissue and cell proteins using 2-D gels stems from the difficulty to resolve and quantify most of the many thousands of protein forms in a typical mammalian cell population or tissue, particularly proteins of low abundance. Selective enrichment for subsets of proteins of interest prior to their separation may overcome some of the limitations of current protein separation methods. However if the goal is the comprehensive analysis of cell and tissue proteins, the separate analysis of protein subsets is tedious and cumbersome and may not be readily accomplished.

Various limitations of 2-D gels have led to the development of other separation strategies in which proteins are separated in two dimensions whereby either the first or second of both separation dimensions differ from those of standard 2-D gel analysis and may include liquid chromatography based separations or liquid electrophoresis. Yet other ways to achieve identification of large numbers of proteins have relied on the digestion of partially separated proteins into peptides followed by separation of peptide digests in one, two or multiple dimensions.

The present invention provides systems and methods for separation of intact proteins, including full proteomes of cells or tissues. Separation modes may include any one of many affinity based separations, any one of many liquid chromatography based separations including different types of ion exchange chromatography and other types of chromatography, any one of many gel based separations, any one of many electrophoretic based separations including gel electrophoresis, liquid electrophoresis, capillary electrophoresis etc. Certain configurations of the separation modes allow for true 3-D separation. Experiments conducted during the course of the development of the present invention have identified configurations that achieve improved separations compared to standard 2-D analysis. The systems and methods of the present invention allow protein separation without sacrificing protein yield due to losses such that at the end fewer proteins are quantified and identified than with a 2-D system.

The present invention provides a novel multidimensional (e.g., three-dimensional (3-D)) proteomics separation system that achieves substantially greater protein separation and identification than is achievable with standard 2-D gels. One of the advantages of the 3-D system of the present invention is that it maintains the ability to characterize the separated proteins based on their pI and molecular weight, while achieving increased resolution. In some embodiments, the three dimensional system consists of first separating proteins based on their charge, followed by separating partially resolved protein fractions individually, based on their hydrophobicity, followed by a third dimension separation of individual fractions, based on protein size. Thus the 3-D system expands the two separation modes of standard 2-D separations (charge and molecular weight) by introducing a separation step based on hydrophobicity. The three separation modes can be accomplished consecutively, using for each separation any one of several procedures that separate proteins based on the principle for that dimension separation. The methods of the present invention provide the added advantage of resolving intact protein, allowing for further analysis of the 3-D) separated proteins.

The present invention further provides an apparatus for performing the separation methods of the present invention, as well as methods of characterizing proteins separated using the methods of the present invention.

Definitions

To facilitate an understanding of the present invention, a number of terms and phrases are defined below:

As used herein, the term “separated protein fraction” refers to one fraction of a sample separated in one or more dimensions (e.g., a fraction from an electrophoresis or chromatography separation).

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the terms “a first separation device configured for separation of said polypeptides in said sample based on charge” and “protein charge separation device” refer to any device that is able to separate proteins in a mixture based on their charge. Exemplary protein charge separation devices include, but are not limited to, “an isoelectric focusing gel electrophoresis device,” “a free-flow electrophoresis device,” “a rotofor electrophoresis device,” and “an ion exchange chromatography device.”

As used herein, the terms “a second separation device configured for separation of said polypeptides in said sample based on hydrophobicity” and “protein hydrophobicity separation device” refer to any device that is able to separate proteins in a mixture based on their hydrophobicity. Exemplary protein hydrophobicity separation devices include, but are not limited to, “a reverse phase chromatography device,” and “a hydrophobic interaction chromatography device.”

As used herein, the terms “a third separation device configured for separation of said polypeptides in said sample based on size” and “protein size separation device” refer to any device that is able to separate proteins in a mixture based on their size. Exemplary protein size separation devices include, but are not limited to, “an SDS-gel electrophoresis device, “a size exclusion chromatography device,” and “a capillary electrophoresis device.”

As used herein, the term “functional assay” refers to any assay that detects an activity of a protein or enzyme. Exemplary activities that can be detected using a functional assay include, but are not limited to, binding to another protein (e.g., an antibody), enzymatic activities, and signaling activities.

As used herein, the term “detection device” refers to any device that can detect the presence of a protein in a sample (e.g., a fraction from a separation device). Exemplary detection devices include, but are not limited to, “a UV/VIS spectrophotometer,” “a fluorescence spectrophotometer,” and “a mass spectrometer.” In some embodiments, the detection device further determines one or more physical properties of the detected proteins (e.g., mass). In some embodiments, two or more detection devices are combined (e.g., fluorescence spectrophotometer and mass spectrometer) to detect and characterize a protein sample.

As used herein, the terms “centralized control system” or “centralized control network” refer to information and equipment management systems (e.g., a computer processor and computer memory) operably linked to multiple devices or apparatus (e.g., chromatography or electrophoresis apparatuses). In preferred embodiments, the centralized control network is configured to control the operations of the apparatuses linked to the network. For example, in some embodiments, the centralized control network controls the operation of multiple chromatography or electrophoresis apparatuses, the transfer of sample between the apparatuses, and the analysis and presentation of data.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (MRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using any suitable screening methods. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to systems, apparatuses and methods for multidimensional protein separation. The systems and methods of the present invention are suitable for separating samples containing a large number of unique proteins (e.g., whole cell extracts). However, the methods of the present invention are not limited to the separation of whole cell extracts. The methods of the present invention are suitable for use in the analysis of any sample comprising a protein mixture. In particular, the present invention relates to 3-dimension protein separation and characterization methods. In some embodiments, the present invention provides an apparatus for performing multidimensional protein separation methods. In some further embodiments, the present invention provides methods for the further characterization of separated proteins.

I. Multidimensional Separation Methods

In some embodiments, the present invention provides multidimensional (e.g., 3-D) protein separation systems and methods. Initial experiments conducted during the course of development of the present invention resulted in the conclusion that use of protein size based separations in the first dimension achieved poor resolution of complex mixtures such that a unique form of a protein was localized to a relatively large number of fractions, creating difficulty in achieving adequate resolution in the three-dimensional separation system. Further experiments indicated that the use of reverse phase chromatography for the first dimension separation was difficult to achieve with conventional columns because of the need to separate large amounts of protein in complex mixtures. Additionally, poor recovery of proteins off the column was observed.

Further experiments conducted during the course of development of the present invention led to the discovery that a functional order of the 3-D separation is separation based on charge (1^{st} dimension), separation based on hydrophobicity (2^{nd} dimension) and separation based on size (3^{rd} dimension). The use of size based separations in the third dimension was found to be particularly advantageous given the ability to achieve a high level of multiplexing with size based separations.

A. First Dimension

In preferred embodiments, the first separation dimension is a separation based on protein charge. Proteins may be separated using any method that provides separation based on charge. In preferred embodiments, the first separation dimension allows for the rapid analysis of large quantities of protein. It is further preferred that the first dimension separation maintain large amounts of protein in a soluble state without precipitation. Additionally preferred first dimension separation steps provide sufficient resolution such that a unique form of a protein is not spread out into a relatively large proportion of the fractions collected following completion of the first dimension separation. It is also preferred that the first dimension separation is compatible with the second dimension separation (e.g., buffer and ionic strength conditions).

Exemplary first dimension separation methods include, but are not limited to, electrophoresis using procedures such as free flow electrophoresis or isoelectric focusing slab gel, liquid electrophoresis using carrier ampholytes, and liquid chromatography using ion exchange separation.

1. Gel Based Methods

In some embodiments, gel-based separation methods are utilized in the first dimension separation based on charge. Exemplary gel-based methods include, but are not limited to, those disclosed below.

a) Carrier Ampholyte Based Slab Gel IEF Separation with a Whole Gel Eluter

In some embodiments, an ampholyte based slab gel is used for IEF separation of protein fractions. In this case the protein solution is loaded onto a slab gel and the proteins separate in to a series of gel-wide bands containing proteins of the same pI. These proteins are then harvested using, e.g., a whole gel eluter (WGE, from Biorad). This type of gel can be loaded with up to 20 mg of protein.

b) IPG Slab Gel IEF Separation with a Whole Gel Eluter

In other embodiments, an immobiline gradient slab gel is utilized. Here the proteins are loaded onto a immobiline pI gradient slab gel and separated into a series of gel-wide bands containing proteins of the same pI. These proteins are electro-eluted using, e.g., the WGE. The IPG gel can be loaded with at least 60 mg of protein.

2. Liquid Electrophoresis

In other embodiments, the first separation dimension utilizes liquid electrophoresis to separate proteins based on charge.

a) Free-Flow Electrophoresis

In some embodiments, the liquid electrophoresis separation is free-flow electrophoresis. Free-flow electrophoresis employs the continuous flow of a replenished buffer between two narrowly spaced plates in the presence of a DC electric field that is applied in the plane of the bounding plates transverse to the direction of fluid flow (See e.g., U.S. Pat. Nos. 6,387,707; 6,328,869, 6,328,868; each of which is herein incorporated by reference). As they traverse the electric field, charged proteins are deflected in proportion to their electrophoretic mobility and collected in separate outlets for subsequent analysis. In contrast to conventional electrophoresis, free-flow electrophoresis is a continuous process with high throughput and it requires no supporting medium such as a gel.

b) Rotofor

In other embodiments, the liquid electrophoresis separation is Rotofor separation. This device (Biorad) separates proteins in the liquid phase according to their pI (See e.g., Ayala et al., Appl. Biochem. Biotech. 69:11 [1998]). This device allows for high protein loading and rapid separations that require only four to six hours to perform. Proteins are harvested into after a 5-hour IEF separation. This device can be loaded with up to 1 g of protein.

3. Liquid Chromatography

In still further embodiments, liquid chromatography is utilized for the first dimension separation. In some embodiments, ion exchange chromatography is utilized. In ion exchange chromatography, proteins are loaded onto columns containing either cation or anion exchange media at a low ionic strength. A gradient of increasing ionic strength is applied to the column to elute proteins. In some embodiments, ion exchange is performed using HPLC (high pressure liquid chromatography) systems. The amount of material that can be loaded on an ion exchange column depends on the size and type of the column and the chromatography system. In preferred embodiments, preparative ion exchange columns that provide for the loading of large amounts of protein extracts are utilized.

B. Second Dimension

In some preferred embodiments, fractions obtained in a first separation dimension are further separated in a second dimension. In some embodiments, the second dimension separation is a separation based on hydrophobicity. Preferred second dimension separation methods are compatible with the first dimension separation, are able to concentrate fractions from the first dimension such that they are retained tightly retained in the second dimension separation system prior to the start of the second dimension separation, are able to resolve and recover applied proteins following the second dimension separation, are able to achieve a reliable and reproducible separation profile, and are compatible with the third dimension separation system.

Exemplary separation methods for separation based on hydrophobicity include, but are not limited to, reversed phase chromatography and hydrophobic interaction chromatography.

a) Reversed-Phase Chromatography

In some embodiments, second dimension hydrophobic separations utilize reversed phase chromatography. Reversed phase chromatography (RPC) is a separation technique that separates molecules according to hydrophobicity. The RPC medium is highly substituted with hydrocarbon chains, making it very hydrophobic. Proteins, peptides and oligonucleotides adsorb to the hydrocarbon chains even in pure water. Elution uses gradients of increasing concentrations of polar (hydrophobic) organic solvents (e.g., acetonitrile or methanol) in water. Polar proteins will elute first from the column.

In other embodiments, non-porous reversed phase HPLC is used as the hydrophobic separation technique (See e.g., Liang et al., Rap. Comm. Mass Spec., 10:1219 [1996]; Griffin et al., Rap. Comm. Mass Spec., 9:1546 [1995]; Opiteck et al., Anal. Biochem. 258:344 [1998]; Nilsson et al., Rap. Comm. Mass Spec., 11:610 [1997]; Chen et al., Rap. Comm. Mass Spec., 12:1994 [1998]; Wall et al., Anal. Chem., 71:3894 [1999]; Chong et al., Rap. Comm. Mass Spec., 13:1808 [1999]).

b) Hydrophobic Interaction Chromatography

In other embodiments, the second separation dimension utilizes hydrophobic interaction chromatography. Hydrophobic interaction chromatography (HIC) is a liquid chromatography technique that separates biomolecules according to hydrophobicity. HIC medium are typically less hydrophobic that reverse-phase chromatography mediums. High salt concentrations are needed to adsorb the sample to the HIC gel. A gradient of decreasing salt concentration is used to elute proteins.

C. Third Dimension

In some preferred embodiments, protein fractions obtained after the first and second dimension separations are further resolved in a third dimension. In some embodiments, the third dimension separation is a separation based on protein size separation principles. Preferred third dimension separation methods are able to analyze the large numbers of fractions generated in the first and second dimension separations. In preferred embodiments, the third dimension separation method is able to analyze at least 400, preferably at least 800, and even more preferably, at least 1200 samples in a short time span. In preferred embodiments, the samples are analyzed simultaneously. It is also preferred that the third dimension separation methods are compatible with the second dimension separation, are able to resolve protein mixtures of limited complexity into individual protein forms that are recoverable from the separation medium, are able to achieve rapid separation of individual fractions, and are able to efficiently multiplex the separation process, given the large number of fraction that need to be processed.

Exemplary third dimension separation methods include, but are not limited to, SDS gel electrophoresis, capillary electrophoresis, size exclusion chromatography and microfluidics based separation systems.

a) SDS Gel Electrophoresis

In some embodiments, the size-based separation is SDS-polyacrylamide gel electrophoresis. SDS gel electrophoresis utilizes a polyacrylamide gel containing SDS. The SDS denatures proteins and allows for separation based on MW, rather than three-dimensional structure of the folded proteins. Smaller proteins migrate through the gel faster. Exemplary techniques for performing SDS-PAGE are well known in the art (See e.g., J. Sambrook et al., Molecular Cloning: A Laboratory Manual, Cold Spring Harbor Press, NY, pp 9.31-9.58 [1989]).

b) Size Exclusion Chromatography

In other embodiments, size exclusion (or gel filtration) chromatography is used as the size separation method. Gel filtration separates molecules according to size. Within the fractionation range chosen, molecules are eluted in order of decreasing size. The fractionation range depends on the pore sizes of the column medium. Gel filtration media contain pores allowing the sample molecules to penetrate into the gel filtration beads to different degrees depending on size. The ability of gel filtration to separate molecules according to size resides with the highly porous structure of gel filtration media and is based on accessible volume of the chromatography medium.

c) Capillary Electrophoresis

In some preferred embodiments, capillary electrophoresis methods are utilized in the third dimension separation. Capillary electrophoresis provides the advantages of speed and the ability to perform parallel analysis of the large number of fractions obtained from 1^{st} and 2^{nd} dimension separations. For example, in some embodiments of the present invention (e.g., those utilizing a separation apparatus of the present invention), larger tubing and parallel lines of electrophoresis tubing are utilized to handle the large numbers of fractions and protein volume in the final analysis step. For example, in some embodiments, dozens of parallel columns are provided in an apparatus to allow parallel separation of an entire proteome.

II. Apparatus for 3-D Separation

In some embodiments, the present invention provides an apparatus for performing 3-D protein separations. In preferred embodiments, the apparatus is able to analyze large enough quantities of protein to permit recovery and further analysis of separated protein fractions. For example, an exemplary analysis of the proteome of an entire cell may result in 200, 400, or even 1000 fractions after two separation steps. Thus, the initial separation steps are optimized to separate large quantities of proteins and the final separation steps are optimized to separate large number of fractions. In some embodiments, the apparatus is configured for further analysis steps (e.g., MS analysis).

A. Separation

In some embodiments, the present invention features an apparatus and methods for the rapid and efficient separation of proteins. The apparatus includes a sample input device, a first separation device, a first transport device for transporting fractions from the first separation device to the second separation device, a second separation device, a second transport device for transporting fractions from the second separation device to the third separation device, and a third separation device.

In some embodiment, the apparatus of the present invention is configured for processing of complex mixture containing protein amounts ranging from mg quantities to microgram quantities. In some embodiments, the first dimension separation is a preparative type of separation that is performed only once for a given sample. However, it is contemplated that apparatus is configured for multiple second dimension separations and third dimension separations to be performed in parallel. As the number of fractions increases from first to second and third dimension separation, the scale of separation shifts from a preparative level to the analytical level (e.g., using microfluidic devices).

The present invention is not limited to particular separation devices. Any suitable separation device may be used for each separation step, including, but not limited to, those described above. Preferred separation devices are those that are able to rapidly analyze large quantities of mixtures (e.g., cell extracts) comprising a large number of different proteins.

In preferred embodiments, the apparatus further includes control means in communication with the transport and separation devices for controlling the operation of the separation devices and the transport devices.

In some embodiments, the apparatus further comprises additional separation and transport devices for performing additional separation steps (e.g., to perform 4-D, 5-D, etc. separation). The present invention is not limited to particular separation methods for use in the additional separation dimensions. Any suitable device maybe utilized including, but not limited to, those described above. In other embodiments, the fourth dimension separation is performed by a mass spectrometer (See below description of MS).

B. Detection

In some embodiments, the apparatus further comprises a detection device in communication with the third, and optionally the first and/or second separation devices for detection of separated proteins. In some embodiments, the detection is absorbance based. For example, in some embodiments, proteins in the final fractions from the third separation device are detected by a UV/VIS spectrophotometer. The presence of an eluting protein alters the UV absorbance (e.g., at 280 nm) of the solution. The absorbance is recorded (e.g., by a computer) and a trace of the peaks corresponding to protein elution is generated.

In other embodiments, detection is fluorescence based. In some embodiments, the fluorescence of amino acids with natural fluorescence is detected. In preferred embodiments, protein fractions are labeled with a fluorescent dye (e.g., Cy3 or Cy5) and the fluorescence of the dye is detected.

In yet other embodiments, the detection is nuclear magnetic resonance (NMR) based. In still further embodiments, the detection is immunological (e.g., ELISA or RIA). In some embodiments, separated proteins are transferred to a microarray for detection (See below description of characterization methods).

In some embodiments, the detector is designed for the rapid or simultaneous detection of a plurality of eluting fractions. For example, in some embodiments, the final separation step is capillary electrophoresis and the separation device comprises a plurality of parallel capillaries (e.g., greater than 20, preferably greater than 50, even more preferably greater than 200, and still more preferably, greater than 1000). In some embodiments, the detector is a scanning detector that rapidly records signal (e.g., UVNIS or fluorescence signal) from capillaries. In other embodiments, the detector comprises a series of parallel detectors, with one detector for each capillary. In still further embodiments, the detector is a hybrid of the scanning and the dedicated detector. For example, in some embodiments, the detector may have one detector for every 2 or more capillaries and scan the two or more capillaries.

In still further embodiments, the detector is a device for performing mass spectroscopy analysis of the separated protein fractions. Mass spectroscopy (MS) allows for the determination of the mass and/or identity of proteins (See e.g., below description of mass spectroscopy).

III. The Present Invention in Operation

In some embodiments, following protein separation and detection (e.g., using the protein separation apparatus of the present invention) separated proteins are further characterized.

A. Further Separation Steps

In some embodiments, following separation in three dimensions, as described above, proteins are further separated and characterized using mass spectroscopy. Exemplary Mass spectroscopy methods include, but are not limited to, MALDI-TOF-MS (U.S. Pat. Nos. 6,387,628 and 6,281,493, each of which is herein incorporated by reference); ESI oa TOF (LCT, Micromass) (See e.g., U.S. Pat. No. 6,002,127, herein incorporated by reference); ion trap mass spectrometry (U.S. Pat. Nos. 5,572,025, 5,696,376, 5,399,857, 5,420,425, each of which is herein incorporated by reference); ion trap/time-of-flight mass spectrometry; quadrupole and triple quadrupole mass spectrometry (U.S. Pat. No. 5,789,747, herein incorporated by reference); Fourier Transform (ICR) mass spectrometry (U.S. Pat. Nos. 3,937,955 and 4,755,670, each of which is herein incorporated by reference); and magnetic sector mass spectrometry.

In some embodiments (e.g., where the final separation step is a gel electrophoresis separation), proteins may first be eluted from the gel before MS is performed. Protein samples may be eluted from the gel using any suitable method. In some embodiments, (e.g., where automated apparatuses are used), gel extraction is automated. In the case of a liquid separation in the final dimension, samples can be directly transferred to the mass spectrophotometer for analysis using any suitable method.

B. Characterization

In some embodiments, the separation and characterization methods of the present invention are used to compare two samples (e.g., a cancer sample and a normal sample derived from the same organ or cell type). For example, in some embodiments, proteins from one sample are labeled with a dye that fluoresces a particular color (e.g., Cy3) and protein from the second sample are labeled with a dye that fluoresces a second color (e.g., Cy5). Following separation, the samples are mapped (e.g., using computer software) and proteins with a different expression level are identified. In some embodiments, the separated proteins are mixed prior to their addition to the separation apparatus.

In still further embodiments, following separation, arrays of proteins are attached to a solid support. In preferred embodiments, proteins or fractions are addressed to a specific location on the solid support (e.g., as identified by fraction number). Proteins may be attached to the array surface using any suitable method (See e.g., U.S. Pat. No. 6,406,921, U.S. patent applications 20020110933A1, 20020102617, WO 01/68671, and WO 00/54046; each of which is herein incorporated by reference). Once protein are attached to the array, they can be screened for a desired property (e.g., binding to a specific protein) using any suitable functional assay.

For example, in one exemplary embodiment, the methods of the present invention are used to identify tumor antigens (e.g., proteins that elicit an immune response in cancerous tissues, but not normal tissues). Protein samples are separated using the separation methods of the present invention. Following separation, the separated fractions are transferred to an array. The array is then contacted with serum from a subject with cancer, and as a control, serum from a healthy subject. Antibodies present in the serum are then allowed to bind to the array. The serum is washed from the array. The array is next contacted with an antibody that binds to any IgG, thus binding to antibodies that have bound to tumor antigens. Proteins that are bound by antibodies in the cancer serum, but not the normal serum, are tumor antigens. Due to the addressable nature of the arrays of the present invention, the proteins identified as tumor antigens can be easily identified for further analysis (e.g., identification using mass spectroscopy).

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and ° C. (degrees Centigrade).

### EXAMPLE 1

3-D Separation of Cell Culture Lysates

Cell lysates from A549 cells, LOVO cells, and SY5Y cell were separated in three dimensions. The first dimension was ion exchange chromatography or liquid isoelectric focusing. Ion exchange chromatography was performed using a column of 4.6 mm I.D. X 150 150 mmL at a flow rate of 0.8 ml/min. Buffer A was 10 mmol/L NH₄OAc, pH 8.0 and buffer B was 2 mol/L NH₄OAc, pH 7.50. The gradient was as follows: 0-5 min, 100% A; 5-45 min, 100-50% A; 45-60 min, 50-5% A.

Isoelectric focusing was performed using a column of 4.6 mm I.D.×150 mmL, at at flow rate of 1.4 ml/min in a running buffer of 6 mol/L urea, 2 mol/L thiourea, 1.0% n-octyl β-D-glucopyranoside, 2 mmol (w/v) dithierythritol (DTE) and 2% (w/v) ampholytes. 80 ml of running buffer was added to the focusing chamber and pre-focused at 12 Watt for 1 hr. 300 μl of protein sample was mixed with 700 μl of running buffer and votexed for 30 s, and loaded into the focusing chamber. The column was run at 12 Watt for 5 hrs. 20 fractions were harvested into separated vials by using a vacuum source attached through plastic tubing to an array of 20 needles. The fractions were stored at −80° C. for further use.

The second dimension separation was reverse phase chromatography using a column of 508 μm I.D.×200 mmL at a flow rate of 50 μl/min. The reversed phase buffer was A: 98% H₂O+2% ACN+0.1% TFA; B: 10% H₂O+90% ACN+0.1% TFA. The gradient was as follows: 0-2.5 min, 95% A; 2.5-5 min, 95-75% A; 5-45 min, 75-35% A; 45-50 min, 35% A; 50-55 min, 35-15% A; 55-60 min, 15-5% A.

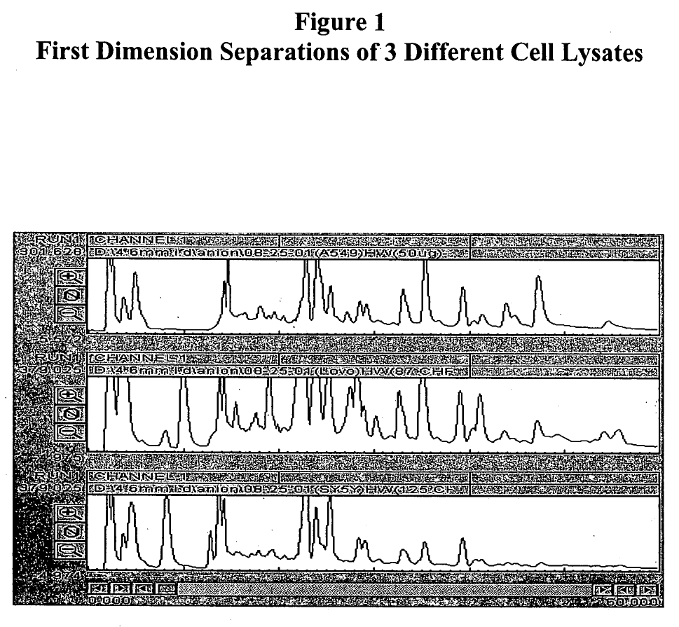

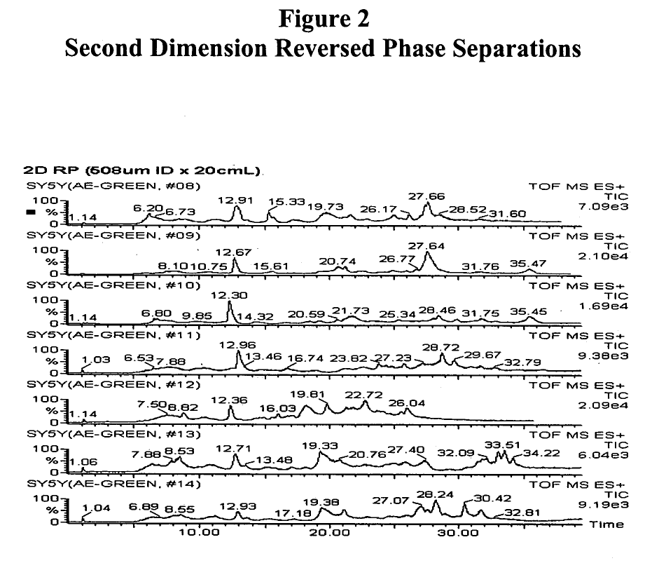

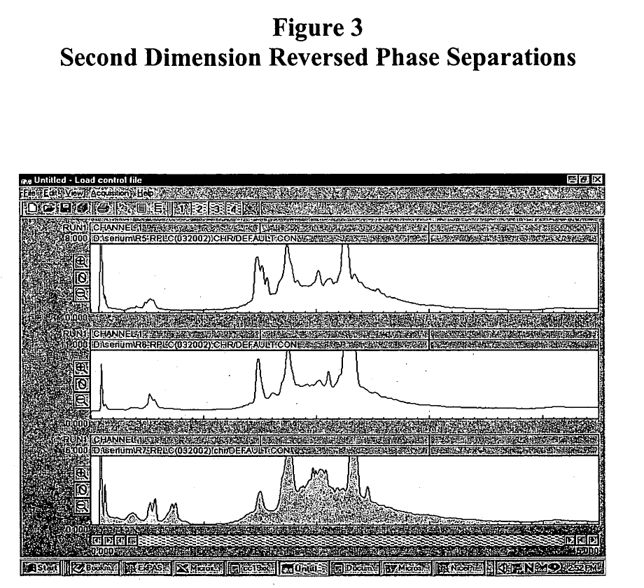

FIG. 1 shows the results of the first dimension ion exchange separation. FIG. 2 shows the results of second dimension reversed phase separation of individual fractions from a first dimension ion exchange separation. FIG. 3 shows the results of second dimension reversed phase separation of individual fractions from a first dimension isoelectric focusing separation.

### EXAMPLE 2

Separation of Plasma Proteins

Plasma proteins were separated in three dimensions: isoelectric focusing (first dimension), reverse phase chromatography (second dimension) and SDS-PAGE (third dimension). Isoelectric focusing and reverse phase chromatography were performed as described in Example 2. SDS-PAGE was performed with a slab gel of 18 cmL×16 cmW; spacer, 1 mm. Mass spectroscopy was performed using a NanoFlow capillary HPLC-Q-TOF micro (MicroMass, Manchester, UK). Peptide sequences were obtained by SurveyScan, MS/MS (m/z: 80-1900) and searched against SwissProt protein sequence database using proteinLynx Global Server (available on the Internet Web site of Micromass). MALDI-TOF/MS perseptive Voyager Biospectrometry Workstation (PerSeptive Biosystem, Framingham, Mass., USA), peptide mass fingerprint (PMF) was used to search against SwissProt protein sequence database using the MS-Fit database searching engine (available on the Internet Web site of Univ. of CA, San Fransisco).

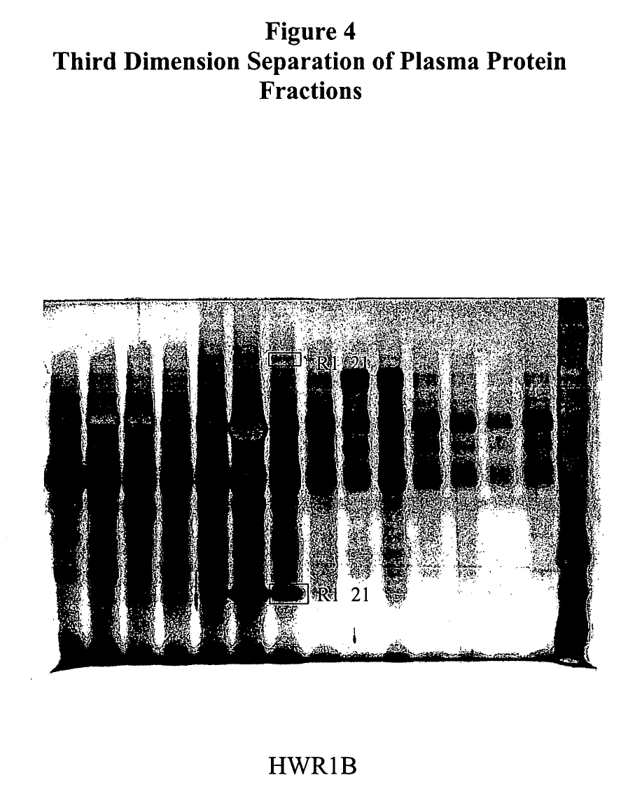

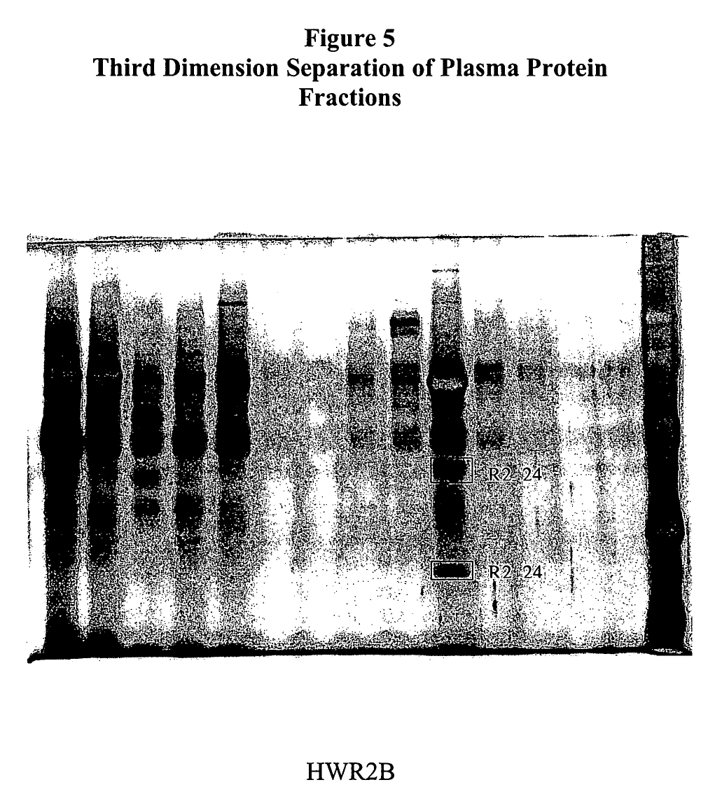

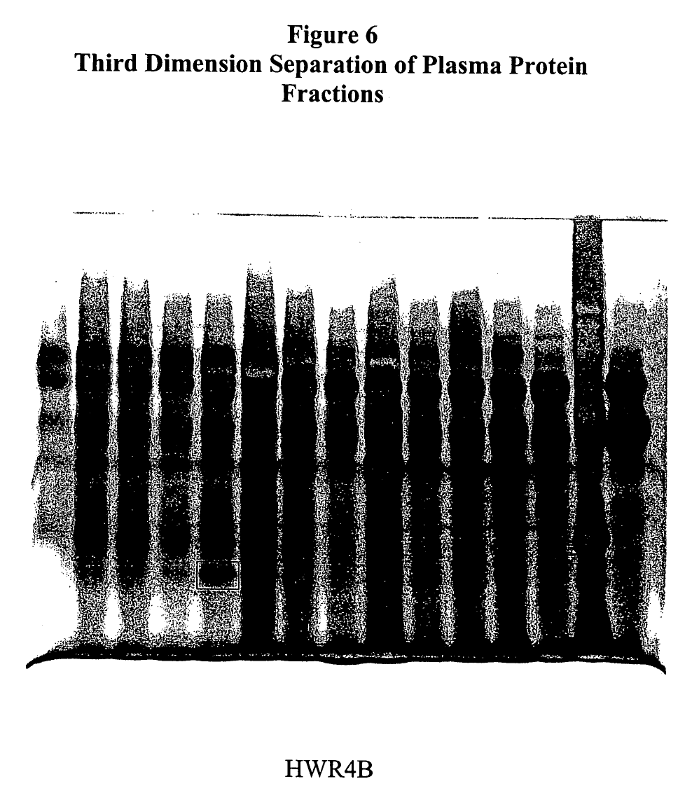

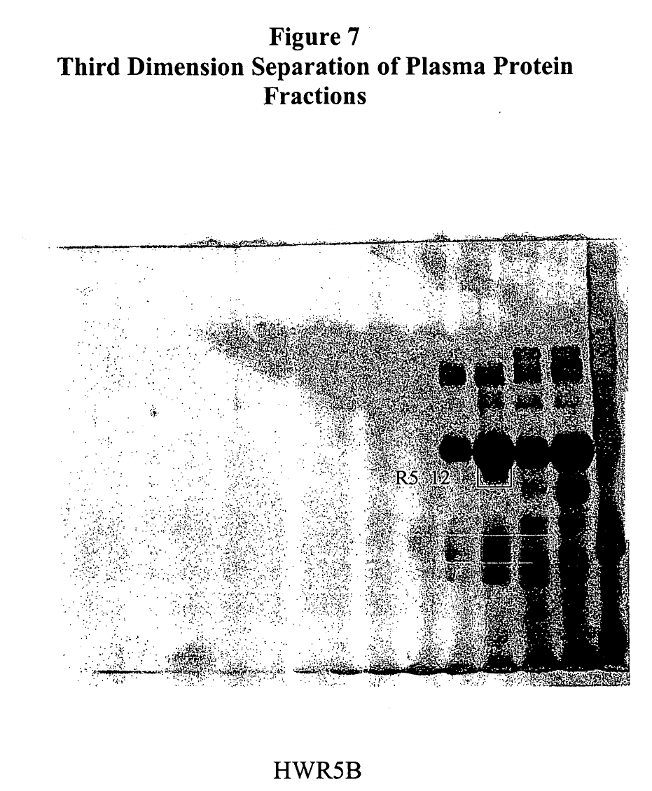

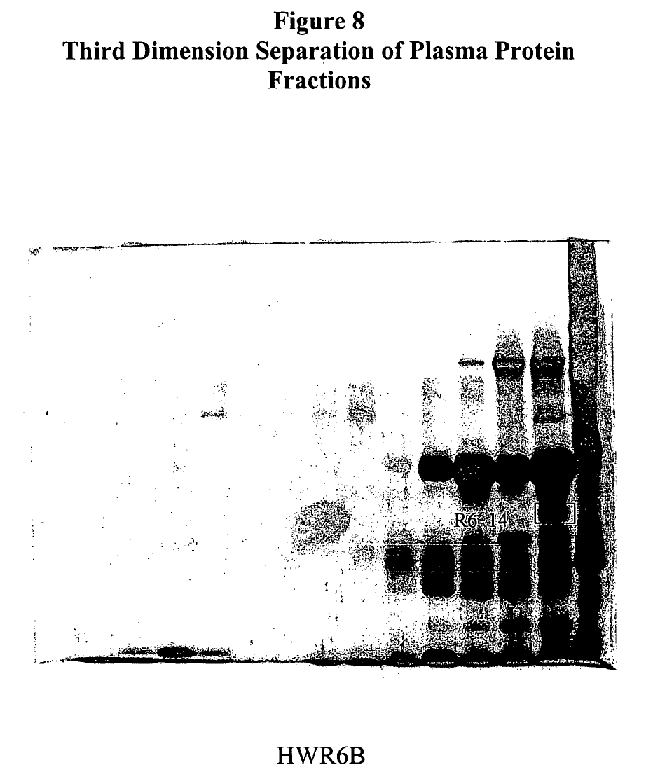

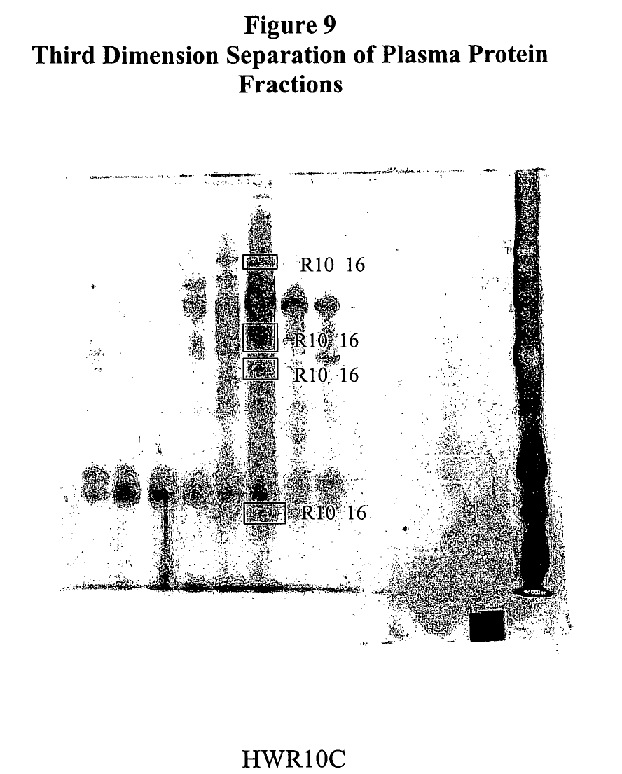

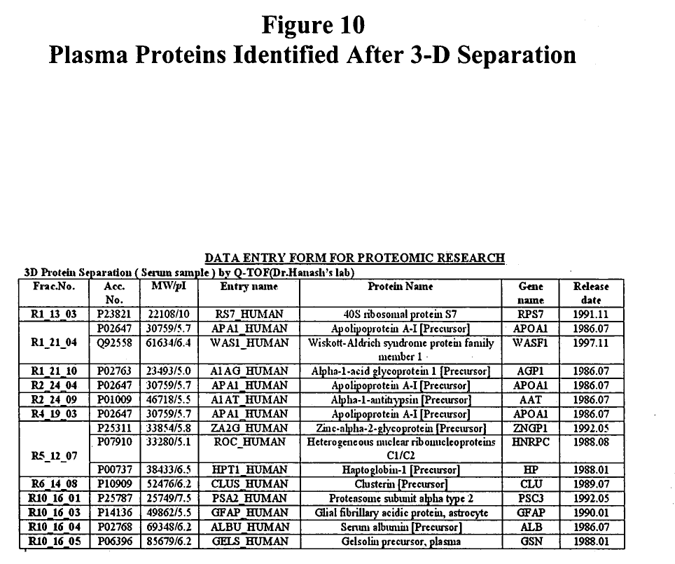

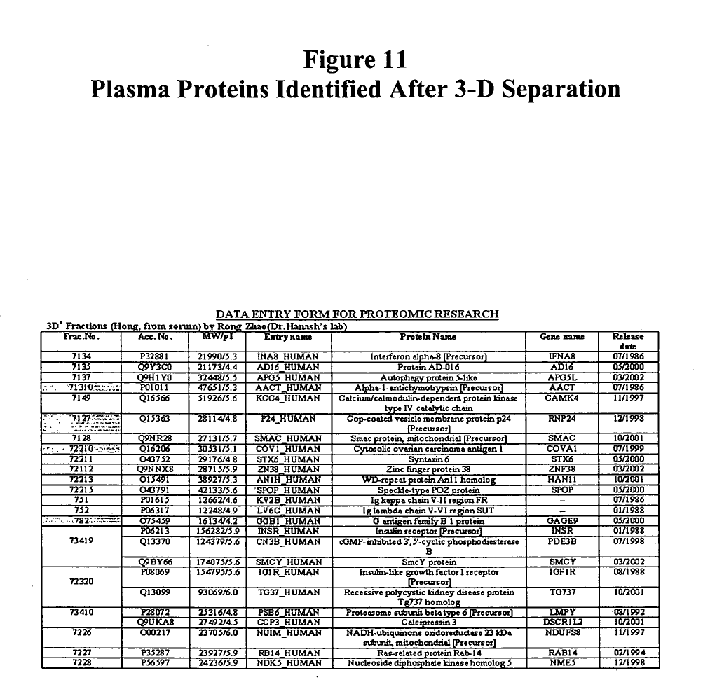

Exemplary SDS-PAGE gels are shown in FIGS. 4-9. FIGS. 10-11 show examples of proteins from 3D separation that were identified using Mass Spectroscopy. This example demonstrates that the combination of the three separation modes applied to intact, undigested plasma proteins achieved substantial resolution and is compatible with identification of resolved proteins using standard procedures such as mass spectrometry.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method, comprising:
a) providing
i) a sample comprising a plurality of polypeptides;
ii) a first separation device configured for separation of said polypeptides in said sample based on charge;
iii) a second separation device configure for separation of said polypeptides is said sample based on hydrophobicity; and
iv) a third separation device configured for separation of said polypeptides in said sample based on size; and
b) separating said sample with said first separation device to generate a charge separated protein sample, wherein said charge separated sample comprises a plurality of fractions;
c) separating said charge separated sample with said second separation device to generate a charge and hydrophobicity separated sample, wherein said charge and hydrophobicity separated sample comprises a plurality of fractions; and
d) separating said charge and hydrophobicity separated sample with said third separation device to generate a charge, hydrophobicity, and size separated sample, wherein said charge, hydrophobicity and size separated sample comprises a plurality of fractions.

2. The method of claim 1, wherein said first separation device is configured for performing a separation technique selected from the group consisting of isoelectric focusing gel electrophoresis, free-flow electrophoresis, rotofor electrophoresis and ion exchange chromatography.

3. The method of claim 1, wherein said second separation device is configured for performing a separation technique selected from the group consisting of reversed-phase chromatography and hydrophobic interaction chromatography.

4. The method of claim 1, wherein said third separation device is configured for performing a separation technique selected from the group consisting of SDS-gel electrophoresis, size exclusion chromatography, and capillary electrophoresis.

5. The method of claim 1, further comprising the step of detecting polypeptides in said fractions of said charge, hydrophobicity, and size separated sample.

6. The method of claim 5, wherein said detecting comprises a detection method selected from the group consisting of UV/VS spectrophotometry, fluorescence spectrophotometry, and mass spectrometry.

7. The method of claim 6, wherein said mass spectroscopy is selected from the group consisting of MALDI-TOF-MS, ESI oa TOF, ion trap mass spectrometry, ion trap/time-of-flight mass spectrometry; quadrupole mass spectrometry, triple quadrupole mass spectrometry, Fourier Transform (ICR) mass spectrometry, and magnetic sector mass spectrometry.

8. The method of claim 1, further comprising the step of attaching said plurality of fractions of said charge, hydrophobicity, and size separated sample to a solid support.

9. The method of claim 8, wherein said plurality of fractions are arrayed on said solid support.

10. The method of claim 9, further comprising the step of performing a functional assay on said arrayed plurality of fractions.

11. The method of claim 10, wherein said functional assay comprises an antibody binding assay.

12. The method of claim 1, wherein said plurality of polypeptide comprise a proteome.

13. The method of claim 1, further providing a second sample comprising a plurality of polypeptides.

14. The method of claim 13, wherein said sample comprises a proteome of a non-cancerous cell and said second sample comprises a proteome of a cancerous cell.

15. The method of claim 14, further comprising the step of comparing said charge, hydrophobicity, and size separated sample to a charge, hydrophobicity, and size separated second sample.

16. A protein separation apparatus, comprising a first separation device, wherein said first separation device is a protein charge separation device; a second separation device, wherein said second device is a protein hybrophobicity separation device; and a third separation device, wherein said third separation device is a protein size separation device.

17. The apparatus of claim 16, wherein said first separation device is selected from the group consisting of a isoelectric focusing gel electrophoresis device, a free-flow electrophoresis device, a rotofor electrophoresis device, and an ion exchange chromatography device.

18. The apparatus of claim 16, wherein said second separation device is selected from the group consisting of a reversed-phase chromatography device and a hydrophobic interaction chromatography device.

19. The apparatus of claim 16, wherein said third separation device is selected from the group consisting of an SDS-gel electrophoresis device, a size exclusion chromatography device, and a capillary electrophoresis device.

20. The apparatus of claim 16, further comprising a detection device.

21. The apparatus of claim 20, wherein said detection device is selected from the group consisting of a UV/VS spectrophotometer, a fluorescence spectrophotometer, and a mass spectrometer.

22. The apparatus of claim 21, wherein said mass spectrometer is selected from the group consisting of a MALDI-TOF-MS, a ESI oa TOF, an ion trap mass spectrometer, an ion trap/time-of-flight mass spectrometer; a quadrupole mass spectrometer, a triple quadrupole mass spectrometer, a Fourier Transform (ICR) mass spectrometer, and a magnetic sector mass spectrometer.

23. A system comprising a protein separation apparatus, said apparatus comprising a first separation device, wherein said first separation device is a protein charge separation device; a second separation device, wherein said second device is a protein hybrophobicity separation device; and a third separation device, wherein said third separation device is a protein size separation device.

24. The system of claim 23, wherein said first separation device is selected from the group consisting of a isoelectric focusing gel electrophoresis device, a free-flow electrophoresis device, a rotofor electrophoresis device, and an ion exchange chromatography device.

25. The system of claim 23, wherein said second separation device is selected from the group consisting of a reversed-phase chromatography device and a hydrophobic interaction chromatography device.

26. The system of claim 23, wherein said third separation device is selected from the group consisting of an SDS-gel electrophoresis device, a size exclusion chromatography device, and a capillary electrophoresis device.

27. The system of claim 23, where said apparatus further comprises a detection device.

28. The system of claim 27, wherein said detection device is selected from the group consisting of a UV/VS spectrophotometer, a fluorescence spectrophotometer, and a mass spectrometer.

29. The system of claim 28, wherein said mass spectrometer is selected from the group consisting of a MALDI-TOF-MS, a ESI oa TOF, an ion trap mass spectrometer, an ion trap/time-of-flight mass spectrometer; a quadrupole mass spectrometer, a triple quadrupole mass spectrometer, a Fourier Transform (ICR) mass spectrometer, and a magnetic sector mass spectrometer.

30. The system of claim 23, further comprising a protein characterization apparatus in communication with said protein characterization apparatus.

31. The system of claim 30, wherein said protein characterization apparatus is a protein array analysis apparatus.

32. The system of claim 31, wherein said protein array analysis apparatus is configured for performing a functional assay on a separated protein sample.

33. The system of claim 32, wherein said functional assay is an antibody binding assay.
